(19) United States
(12) Patent Application Publication
(10) Pub. No.: US 2023/0059124 A1
(43) Pub. Date: Feb. 23, 2023

(54) Novel TAS2R38 Bitter Taste Receptor Agonist

(71) Applicant: Eric H Kuhrts, Fairfield, CA (US)

(72) Inventor: Eric H Kuhrts, Fairfield, CA (US)

(21) Appl. No.: 17/812,960

(22) Filed: Jul. 15, 2022

Related U.S. Application Data
(60) Provisional application No. 63/223,659, filed on Jul. 20, 2021.

(51) Int. Cl. A61K 31/05; A61P 37/04; A61K 45/06

(52) U.S. Cl. CPC A61K 31/05; A61P 37/04; A61K 45/06

(57) ABSTRACT

A method for simulating the activity of the bitter taste receptor TAS2R38 using a prenylflavonoid such as xanthohumol, or an extract thereof. A method of stimulating the innate immune system and prevention of upper airway infections in the body can include a prenylflavonoid extract such as xanthohumol in a solubilized system such as a spray, mouth rinse, beverage, fast dissolving dosage form, or any other means of making contact of the molecule with the receptor in the mouth or nasal passages. Also included is a method for lengthening the lifespan of humans or animals by stimulating the TAS2R38 taste receptor with the prenylflavonoid xanthohumol.

The present application claims the benefit of U.S. Provisional Patent Application No. 63/223,659, which was filed on Jul. 20, 2021, the entirety of which is incorporated herein by reference.

### BACKGROUND

The sweet and bitter taste receptors, known as T1Rs and T2Rs respectively, are G-protein-coupled receptors that were first discovered on the tongue in type 2 taste cells of the taste bud. Receptors are cellular proteins which when activated, cause the cell to modify its activity, resulting in certain changes. T2Rs and T1Rs have been identified in various other organ systems including the upper and lower respiratory airway, oral cavity, thyroid, lung, heart, brain, urinary, and digestive tracts. More recently, functional taste receptors have been found in the skin. There are 25 T2R bitter receptor isoforms found on the tongue.

The role of taste receptors (T2Rs) in other parts of the body other than the tongue is currently under intense investigation, but it is now accepted that taste receptors are involved in various chemosensory functions beyond the originally described brain pathways that originate on the tongue and begin our sensory experience of taste. Surprisingly, functional taste receptors have even been located in human keratinocytes (skin). Specific taste receptors have also been associated with longevity as a result of epidemiological studies conducted in certain ‘blue zones” or areas of the world where people live longer. From these gene studies, certain variants of specific taste receptor genes were found to have a high degree of correlation with longevity. There is significant variability in taste receptors, with many genetic polymorphisms, and this is believed to be the reason for differences in preferences for foods and seasoning of food among different individuals and different countries. Furthermore, taste receptor variations could explain the differences in immune response and the resistance to inhaled pathogens in the airway entry points.

Taste receptors have been identified in the airway ciliated cells of the bronchial epithelium and the sinonasal epithelium. Bitter substances act as agonists for taste receptors (T2Rs), stimulating calcium dependent increase in activity of ciliary cells.

Over the last few years, research has verified a connection between nasal innate immunity and sweet and bitter taste receptors, and it has become possible to target certain specific T2Rs to treat diseases which have entry points in the upper airway, particularly, the sinonasal cavity. As the entry point for many respiratory infections, and the gateway for entry of bacteria, viruses, and other pathogens or invading organisms, the mouth and nasal cavity are the sentinel points for a possible strategy of protection of the body from contagious diseases.

Multiple T2R receptors, T2R4, T2R14, T2R16, and T2R38, in the cilia of sinonasal epithelial cells have been identified. However, one specific taste receptor isoform, TAS2R38 (T2R38), in airway cilia, has been specifically found to be activated by gram-negative bacteria and other invading pathogens. In addition, this receptor has been correlated with longevity in humans from genetic studies conducted in specific parts of the world where people live longer (blue zones). Also, of particular interest is recent research correlating severity of SARS-CoV2, or the Covid 19 virus with the T2R38 taste receptor, and how polymorphisms in the gene can determine the severity of the disease progression, hospitalization, and even death from the virus.

### BRIEF DESCRIPTION OF THE DRAWINGS

FIG. 1 Shows the effect of the prenylflavonoid xanthohumol on bitter taste receptor TAS2R38 gene expression using qPCR (Polymer Chain Reaction).

### DETAILED DESCRIPTION

In humans, bacterial stimulation of cilia-localized taste receptor T2R38 (TAS2R38) leads to rapid intracellular nitric oxide (NO) production that occurs through the activation of calcium-dependent nitric oxide (NO) synthase (NOS) via the endothelial NOS (eNOS) isoform. Inside the cell, the activated nitric oxide then stimulates protein kinase G (PRG) to phosphorylate ciliary proteins, which increases the frequency and motility of ciliary beating, which in turn speeds up mucociliary clearance and transport. Reactive byproducts of NO (nitric oxide) called reactive nitrogen species (RNS) are produced, and these can also damage pathogens, and breakdown bacterial DNA. This system represents a major pathway in the body's defense against bacterial, fungal, and viral infection.

In the airway, taste receptors (T2Rs) are present on ciliated epithelial cells and solitary chemosensory cells (SCC). T2R38, one of the many isoforms of T2Rs, is a receptor that is located in motile cilia in humans. It can be stimulated (activated) by phenylthiocarbamide (PTC) and propylthiouracil (PROP). When T2R38 is stimulated by agonists, nitric oxide (NO) and various peptides are produced, and this causes mucociliary clearance (MCC) which kills pathogens in the human respiratory mucosa.

For purposes of consistency, the nomenclature for the T2R38 bitter taste receptor used herein shall be TAS2R38. Among taste receptors, the bitter taste receptors have been found to be G-protein coupled receptors, which were discovered to play a role in innate immunity by inducing the release of nitric oxide through ciliated epithelial cells. The bitter taste receptor T2R38 (TAS2R38) exists in different polymorphisms, and these have been classified as PAV/PAV for supertasters, PAV/AVI for taster, and AVI/AVI for non-tasters. Individuals who are PAV/PAV or classified as supertasters for this particular taste receptor polymorphism have been found to have less incidences of gram-negative upper respiratory infections and report better quality of life than non-tasters (AVI/AVI).

The expression of T2R38 (TAS2R38) can be determined in an individual by testing with paper test strips using certain chemicals such as phenylthiocarbamide (PTC). Individuals who are homozygotoc for PAV/PAV can taste the bitterness of the chemical in the test strip, but those with homozygotic non-functioning polymorphisms (AVI/AVI) do not taste the intensity of the bitterness to the same degree. The magnitude of the bitter intensity to the senses of certain chemicals is either amplified in certain individuals or decreased in other individuals depending on the phenotypic expression of the specific taste receptor. About 20% of Caucasian are homozygous for the PAV allele. That means they are considered supertasters and are more sensitive to bitterness from specific bitter flavors triggered by chemical compounds such as phenylthiocarbamide (PCT) or 6-propyl-2-thiouracil (PROP) that have been found to stimulate or activate the TAS2R38 taste receptor. Another 30% of Caucasians ae considered to be non-tasters, as they cannot detect the bitterness of these specific compounds. These are homozygous AVI allele individuals. In between, are those who are heterozygous PAV/AVI, and they exhibit a variable degree of sensitivity to these TAS2R38 activating chemical compounds.

Recently, with the world-wide pandemic of SARS-CoV2 (Covid-19), it has been possible to correlate the severity of medical complications, hospitalization, and even death with the specific taste receptor TAS2R38. In addition, otolaryngologists and head and neck surgeons as well as emergency physicians and patients have noticed that patients admitted to the hospital who were experiencing a loss of taste and smell, were more likely to have Covid-19 than other types of infection. Eventually, studies were conducted to determine among patients who were diagnosed with the virus, whether there was any correlation with the severity of the infection and the presence of the particular homozygotic non-functioning polymorphisms. Interestingly, among those hospitalized with severe complications due to the virus, 100% were non-tasters, while 100% of the patients who manifested mild or moderate symptoms were classified as tasters, based on sensitivity to chemical compounds known to activate the TAS2R38 receptor.

Since the emergence of the severe acute respiratory syndrome coronavirus or the SARS-CoV2 and subsequently, the Covid-19 virus pandemic, medical clinicians have noticed the widespread occurrence of loss of taste and smell sensation in patients diagnosed with the disease. SARS-CoV-2 is the causative pathogen of Covid-19. Among Covid-19 patients for example, 83% experienced a loss of taste. Earlier, some clinicians had already noticed rhinovirus, coronavirus, parainfluenza virus, and Epstein-Barr virus in nasal secretion of patients with olfactory disorders, signaling that smell (olfactory) receptors may also be involved in the transmission and invasion of pathogens in the entry points to the body, the nose and mouth. The existence, abundance, and expression of angiotensin-converting enzyme 2 (ACE2) cell receptors on respiratory epithelium and oral mucosa, particularly on the tongue, provides further clues to the importance of taste receptors in the mouth as a primary entry point for invading pathogens. There is clearly a high degree of expression of ACE2 receptors of Covid19 virus on the epithelial cells of the oral mucosa. The virus uses these receptors to enter epithelial cells. In summary, chemosensory dysfunction, especially involving taste and smell is a common occurrence in the majority of patients diagnosed with Covid19 virus, as well as other pathological invading organisms.

If the sinonasal immune defense is impaired, diseases such as chronic rhinosinusitis or CRS may occur. CRS (chronic rhinosinusitis) is a fairly common disease, affecting more than 16 million Americans annually, and results in about 25% of all adult antibiotic prescriptions. Inhaled pathogens, such as bacteria and viruses, toxins, and particulates continuously challenge the respiratory system. The principal physical defense against these inhaled insults is mucociliary clearance (MCC), which has 2 components, mucus production and mucus transport. Coordinated ciliary beating transports debris-laden mucus from both the upper and lower respiratory passages toward the oropharynx, from which it is cleared by expectoration or swallowing. Ciliary beating accelerates in response to multiple host and environmental stimuli through several second messenger pathways including intracellular Ca2+ and NO production. In addition to its role as a second messenger, NO diffuses into the airways where it has antimicrobial properties and is therefore central to host defense against respiratory infections.

The pathogen-derived triggers of respiratory host defenses are likely to include those that humans report to taste bitter, based on recent observations that bitter taste receptors (T2Rs) are expressed in both upper and lower human respiratory epithelium.

Additionally, in vitro stimulation of human lower airway cultures with various bitter-tasting compounds increased intracellular Ca2+ and ciliary beat frequency (CBF). A summary review of research findings related to taste receptors and innate immunity can be found in the following paper: Cary, R.; Lee, R., Taste Receptors in Upper Airway Innate Immunity. Nutrients, 11, 1-17; 2019.

Lastly, recent research has correlated longer lifespan in humans with the “taster variant” (PAV/PAV) of the TAS2R38 receptor gene. The discovery of a natural food type compound that acts as an agonist (a compound that stimulates the activity of the gene) to this receptor in the mouth could represent a novel means of activating the receptor through the diet with the use of a supplement that is not normally contained in food. TAS2R38 can also be found in extra oral tissue, and may have correlations with cystic fibrosis, increase risk of dental caries, colorectal and gastric cancer, and inflammation.

Hops (Humulus lupulis L.) has been used for centuries as a bittering agent in the brewing of beer. Hops contains alpha acids such as humulone, co-humuone, ad-humulone, and beta acids such as lupulone and co-lupulone. Hops also contains many flavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. A typical ethanol extract of hops yields about 3 mg./g (3%) of xanthohumol out of a total flavonoid content of 3.46 mg./g. Dried hop contains about 0.2 to 1.0% by weight xanthohumol.

Xanthohumol and other hop prenylflavonoids have been identified as cancer chemopreventive agents through their interfering action with a variety of cellular mechanisms at low micromolar concentrations such as (1) inhibition of metabolic activation of procarcinogens, (2) induction of carcinogen-detoxifying enzymes, and (3) inhibition of tumor growth by inhibiting inflammatory signals and angiogenesis. There is virtually no xanthohumol in beer. However, the effects of a prenylflavonoid such as xanthohumol have not been tested on taste receptors such as TAS2R38. Up until now, there have been no known natural (botanical) agonists of the TAS2R38 taste receptor that significantly up-regulate the receptor at a reasonable concentration. For purposes of definition, an agonist is a compound that

Ethanol or other solvents may be used to extract higher levels of the prenylflavonoids from hops. The typical prenylflavonoid content of an ethanol extract of hops includes xanthohumol (3 mg/g), desmethylxanthohumol (0.34 mg./g), isoxanthohumol (0.052 mg/g), 6-prenylnaringenin (0.061 mg/g), and 8-prenylnaringenin 0.015 (mg/g). Supercritical carbon dioxide extractions tend to contain much lower levels, or non-existent levels of prenylflavonoids. In fact, these compounds are almost non-existent in standard CO₂ extracts because the prenylflavonoids are virtually insolvent on carbon dioxide. Very little or no xanthohumol is present in beer. Therefore, commercial beer is not a viable source of xanthohumol for therapeutic purposes or for up regulating the TAS2R38 taste receptor. Further extraction and purification enable prenylflavonoids with higher concentrations of up to 98% purity to be produced.

### Quantifying Gene Expression

Recent methods for studying gene expression have developed such as DNA microarray and Quantitative PCR (polymer chain reaction), Real Time Polymer Chain Reaction (qPCR), and Reverse Transcription Polymerase Chain Reaction. A DNA microarray, for example, can be used to measure the expression of a large number of genes simultaneously. This is typically done using a DNA chip (biochip), which is a collection of minute DNA spots attached to a solid surface. The discovery of a compound that increases the activity of a specific gene (an agonist) can be conducted by observing the effects of the compound on a specific gene using these techniques. A compound that stimulates the activity can be said to “up-regulate” the specific gene. To detect an increase in the activity of a specific taste receptor gene such as the TAS2R38 receptor gene, when exposed to a specific molecule, a DNA microarray was first employed, followed by qPCR. Significant up-regulation of the TAS2R38 gene was detected in the DNA microarray study, and further confirmation was found using qPCR. The up regulation of a receptor gene by a chemical compound can also be referred to as an “agonist”. An agonist is a chemical that binds to and activates a biochemical receptor to produce a biological response.

### SUMMARY

The present disclosure is drawn to a method of stimulating the activity of the bitter taste receptor TAS2R38 in the mouth or nasal cavity comprising a solubilized formulation of a prenylaflavonoid such as xanthohumol, a compound found in hops (Humulus lupulus). Hops contains a number of flavonoids, such as xanthohumol, isoxanthohumol, desmethylxanthohumol, 8-prenylnaringenin, and 6-prenylnaringenin. In one example, xanthohumol is from a xanthohumol extract from hops. In another example, the xanthohumol is present at a concentration from about 0.01% to 50% by weight. The formulation can further include an emulsifier, ethanol, d-alpha tocopheryl polyethylene glycol succinate, glycerin, cycoldextrin, phosphatidylcholine, hydrocolloids, PEGylated nanospheres, PLGA nanospheres, or nanoparticles. In another example, there can be a non-ionic surfactant included. In still another example, the prenylflavonoid or xanthohumol can be present as extracts, and the extracts to non-ionic surfactant weight ratio is from about 1:5 to about 1:200.

In another example, a method of stimulating the activity of the TAS2R38 taste receptor can include preparing a solubilized formulation, preferably a water-soluble formulation. The formulation can be administered as a mouth-rinse or spray that can be used to make contact with the receptor in the mouth or nasal passages. The prenylflavonoid or xanthohumol can be formulated with a solubility enhancing agent, such as an emulsifier, ethanol, non-ionic surfactant, d-alpha tocopheryl polyethylene glycol succinate, glycerin, cycoldextrin, phosphatidylcholine, hydrocolloids, PEGylated nanospheres, PLGA nanospheres, or nanoparticles. The formulation can be administered in the form of a water-soluble gel or concentrate. The formulation can include a solid or liquid carrier suitable to form a consumable formulation. When a solid carrier, it can include magnesium carbonate, magnesium stearate, talc, sugar, lactose, pectin, dextrin, starch, gelatin, tragacanth, a low melting wax, cocoa butter, vegetable oil, ethanol, sucrose, mannitol, sorbitol, cellulose, gums, gelatin, collagen, or combination thereof. When a liquid carrier, the consumable formulation can be in the form of a beverage, mouth rinse, or spray. Examples of consumable formulations can include beverages, food, feed, dairy products, yoghurts, gummy bears, lozenges, fortified food, enhanced waters, cereal bars, bakery items, cakes, cookies, dietary supplements, tablets, pills, granules, dragees, capsules, effervescent formulations, non-alcoholic or alcoholic drinks, soft drinks, sport drinks, fruit juices, teas, milk-based drinks, liquid foods, soups, liquid dairy products, or any combination thereof.

In describing and claiming the present invention, the following terminology will be used.

The singular forms “a,” “an,” and “the” include plural referents unless the context clearly dictates otherwise. Thus, for example, reference to “a non-ionic surfactant” includes reference to one or more of such compounds.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, a “prenylflavonoid,” or prenylaflavonoid, refers to a prenylated compound having a substituted or unsubstituted phenol attached to a phenyl via a C3 alkylene substituted with an oxo group. The C3 alkylene may be present in a linear chain arrangement (e.g. a chalcone) or joined with other atoms to form a substituted or unsubstituted ring (e.g. a flavanone). Prenylflavonoids may be derived from natural sources (e.g. hops), or synthesized chemically. A “prenylated” compound refers to those compounds with an attached —CH₂—CH═C(CH₃)₂ group (e.g. geranylated compounds), optionally hydroxylated prenyl tautomers (e.g. —CH₂—CH—C(CH₃)═CH₂, or —CH₂—C(OH)—C(CH₃)═CH₂), and optionally hydroxylated circularized prenyl derivatives having the formula below:

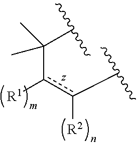

In this formula, the dashed bond z represents a double bond or a single bond. R¹ and R² are independently hydrogen or OH. The symbol [symbol] represents the point of attachment to the remainder of the prenylated compounds.

As used herein, the term “extracted prenylflavonoid” refers to a xanthohumol hops extract that has been extracted by any number of extraction methods including ethanol extractions, supercritical carbon dioxide extractions, or the like. The xanthohumol content, after extraction, will typically be at least 3% wt % pure, but can be 5 wt %, 10 wt %, 30 wt %, 40%, 50%, 80% or even 99 wt % pure.

Hops (Humulus lupulis L.) has been used for centuries as a bittering agent in the brewing of beer. Hops contains alpha acids such as humulone, co-humuone, ad-humulone, and beta acids such as lupulone and co-lupulone. Hops also contains many flavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. A typical ethanol extract of hops yields about 3 mg./g (3%) of xanthohumol out of a total flavonoid content of 3.46 mg./g. Dried hop contains about 0.2 to 1.0% by weight xanthohumol. Xanthohumol, molecular weight is 354.40, CAS 6754-58-1, C21H22O5.

A “non-ionic surfactant,” as used herein, is a surface-active agent that tends to be non-ionized (i.e. uncharged) in neutral solutions (e.g. neutral aqueous solutions).

A “transparent” or “clear” water soluble formulation, as disclosed herein, refers to a formulation that can be clearly seen through with the naked eye and is optionally colored.

An agonist is a chemical that activates a receptor to produce a biological response. Receptors are cellular proteins whose activation causes the cell to modify what it is currently doing. Various scientific techniques have been developed to discover agonists by looking at receptor gene expression. Here, two principal methods have been used for discovery of potential agonists to bitter taste receptors; DNA Microarray and Polymerase Chain Reaction (PCR).

In the present disclosure, unless the context dictates otherwise, all percentages are based on weight.

Concentrations, amounts, solubility, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

With this in mind, in the search for natural compounds or foods that stimulate or increase the activity of the specific taste receptor TAS2R38 (an agonist), it has been found that xanthohumol, a prenylflavonoid derived from the flowers of the hops plant, Humulus lupulus L., has great potential in the treatment of the innate immune system at the entry point for most communicable diseases, the mouth and nasal passages. Furthermore, the same receptor associated with stimulating innate immunity in the upper respiratory tract is also associated with longevity, and possibly other diseases. Epidemiology studies conducted in areas of the world where humans live longer (so called “blue zones”) have demonstrated a correlation with the TAS2R38 bitter taste receptor gene. Individuals with a variant of this gene have longer life spans (Melis, M. et al. TAS2R38 Bitter Taste Receptor and the Attainment of Exceptional Longevity. Nature Scientific Reports. 9, 18047 (2019)) Other beneficial therapeutic properties associated with the activation of the TAS2R38 receptor may be discovered in the future.

In accordance with this, a method of increasing the activity of the TAS2R38 gene, a method of increasing longevity, and a method for the treatment or prevention of upper respiratory infections or prevention from the invasion of various pathogens can comprise administering an extracted hops xanthohumol prenylflavonoid formulation to a subject.

Furthermore, it has now been discovered that the prenylflavonoid xanthohumol from hops, stimulates the activity of the specific taste receptor TAS2R38, which is an unexpected discovery. Additional ingredients or formulations designed to effect the solubility of the prenylflavonoid can also be included, such as the non-ionic surfactants, polyoxyl castor oil, d-alpha tocopheryl polyethylene glycol succinate (1000 succinate), naturally derived surfactants such as saponins from Quillaja Bark, glycerin, cyclodextrin, or beta-cyclodextrin, phosphatidylcholine, hydrocolloids, PEGylated compounds, PLGA nanospheres, nanoparticles, and/or other additives or excipients, some of which may be incorporated to increase the solubility of the prenylaflavonoid such as xanthohumol. In one example, a stable, water-soluble pharmaceutical liquid formulation or composition of the prenylflavonoid xanthohumol is disclosed that can be prepared and administered as described herein. In this method, a water-soluble non-ionic surfactant is heated in a container to a temperature of about 90° F. to about 200° F. while mixing the non-ionic surfactant until a clear non-ionic surfactant is formed. An extracted prenylflavonoid (xanthohumol) is then added to the clear non-ionic surfactant and mixed until a clear non-ionic surfactant-xanthohumol combination is formed so as to constitute from about 20 wt % to 50 wt % surfactant, and from 0.01 wt % to 10 wt % prenylflavonoid (xanthohumol), wherein the prenylflavonoid is sufficiently dispersed or dissolved in the surfactant so that a gel composition is formed containing no visible micelles or particles of prenylflavonoid. In administering this or other formulations to a subject, the resultant water-soluble pharmaceutical gel or concentrate can be administered, or it can be admixed with a liquid or solid carrier for administration.

The prenylflavonoid may be derived from a natural source, such as hops. Hops (Humulus lupulus L.) has been used for centuries as a bittering agent in the brewing of beer. Or it can be synthesized. Hops contain alpha acids such as humulone, co-humulone, ad-humulone, and beta acids such as lupulone and co-lupulone. Hops also contains many prenylflavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. and a molecular weight of 354.4. A typical ethanol extract of hops yields about 3 mg/g (3%) of xanthohumol out of a total flavonoid content of 3.46 mg/g. Dried hop contains about 0.2 to 1.0% by weight xanthohumol. Xanthohumol can be extracted and purified to a concentration of greater than 1 wt %, in certain examples, to greater than 5 wt %, 20 wt %, 40 wt %, 80 wt %, or even 99 wt % pure. By extracting xanthohumol from hops, and formulating into a suitable liquid formulation, spray, beverage, lozenge, food, supplement, or other medicinal dosage form, an effective formulation for treating or preventing upper respiratory infections, and prolonging longevity is possible.

Xanthohumol may be isolated from hops through purification, fractionation, or separation methods that are known to those skilled in the art. Ethanol may be used to extract higher levels of the prenylflavonoids from hops. The typical prenylflavonoid content of an ethanol extract of hops includes xanthohumol (3 mg/g), desmethylxanthohumol (0.34 mg/g), isoxanthohumol (0.052 mg/g), 6-prenylnaringenin (0.061 mg/g), and 8-prenylnaringenin 0.015 (mg/g). Supercritical carbon dioxide extractions tend to contain much lower levels, or non-existent levels of prenylflavonoids. In fact, these compounds are almost non-existent in standard CO₂ extracts because the prenylflavonoids are virtually insolvent on carbon dioxide. In the examples provided herein, a xanthohumol extract of purity of greater than 5 wt % has been used. It is noted herein that any method used to isolate prenylflavonoids are referred to herein as “extractions” or “extracted prenylflavonoids, regardless of how the isolation or concentration occurs. In addition, the compound can be synthesized.

Extracted prenylflavonoids that are useful as described herein can include prenylchalcones and/or prenylflavanones. In some embodiments, the prenylflavonoid is selected from xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, and metabolites and/or derivatives thereof. Prenylflavonoids of interest include xanthohumol, xanthohumol metabolites, and derivatives thereof, in extracted form. By extracting the prenylflavonoids and then using the extracted prenylflavonoids to prepare appropriate formulations, a more pure form of these ingredients can be prepared and used at an appropriate concentration for treating these conditions.

In some embodiments, the hops prenylflavonoid xanthohumol can be present in the formulation at a concentration of at least 1%, 5%, 10%, 20%, 25%, 30%, 35%, 45%, 45%, or 50% by weight. In other embodiments the xanthohumol can be present in the pharmaceutical dosage form at a concentration from 0.01%, 0.1%, 1% to 80%, 5% to 50%, 10% to 35%, or 20% to 25% (by weight).

### Solubilizers

Various formulations of prenylflavonoids may be made using solubilizing agents. A preferable formulation is a water-soluble formulation that can be incorporated into a spray or mouth rinse. Cyclodextrins are a family of cyclic oligosaccharides, consisting of a macrocyclic ring of glucose subunits joined by α-1,4 glycosidic bonds. Cyclodextrins are produced from starch by enzymatic conversion. They are used in food, pharmaceutical, drug delivery, and chemical industries, as well as agriculture and environmental engineering.

Cyclodextrins are composed of 5 or more α-D-glucopyranoside units linked 1->4, as in amylose (a fragment of starch). The largest cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape:

α (alpha)-cyclodextrin: 6 glucose subunits

β (beta)-cyclodextrin: 7 glucose subunits

γ (gamma)-cyclodextrin: 8 glucose subunits

Phospholipids are a class of lipids whose molecule has a hydrophilic “head” containing a phosphate group and two hydrophobic “tails” derived from fatty acids, joined by an alcohol residue (usually a glycerol molecule). Phospholipids have been widely used to prepare liposomal, ethosomal and other nanoformulations of topical, oral and parenteral drugs for differing reasons like improved bio-availability, reduced toxicity and increased permeability across membranes. Liposomes are often composed of phosphatidylcholine-enriched phospholipids and may also contain mixed phospholipid chains with surfactant properties. Phospholipids can act as emulsifiers, enabling oils to form a colloid with water. Phospholipids are one of the components of lecithin, which is found in egg yolks, as well as being extracted from soybeans, and is used as a food additive or supplement in many products.

Medium-chain triglycerides (MCTs) are triglycerides with two or three fatty acids having an aliphatic tail of 6-12 carbon atoms. Rich food sources for commercial extraction of MCTs include palm kernel oil and coconut oil. MCTs can be used as emulsifiers to make oil or lipid based emulsions. MCTs can be used in solutions, liquid suspensions and lipid-based drug delivery systems for emulsions, self-emulsifying drug delivery systems, creams, ointments, gels and foams as well as suppositories. MCTs are also suitable for use as solvent and liquid oily lubricant in soft gel capsules.

Hydrocolloids are certain chemicals (mostly polysaccharides and proteins) that are colloidally dispersible in water. By becoming effectively “soluble” they change the rheology of water by raising the viscosity and/or inducing gelation. Useful hydrocolloids include, but are not limited to pectin, guar gum, acacia gum, sodium carboxymethylcellulose etc.

If a water-soluble gel or concentrate is to be formed (and then mixed with water as described herein), useful non-ionic surfactants include, for example, non-ionic water soluble mono-, di-, and tri-glycerides; non-ionic water soluble mono- and di-fatty acid esters of polyethylene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof; naturally derived surfactants such as saponins from Quillaja Bark, soapwort (Gypsophila Sp.) extracts, and chestnut saponin extracts.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), and derivatives thereof.

Non-ionic water soluble mono- and di-fatty acid esters of polyethylene glycol include d-α-tocopheryl polyethylene glycol 1000 succinate (TPGS), polyethylene glycol 660 12-hydroxystearate (SOLUTOL HS 15), polyoxyl oleate and stearate (e.g. PEG 400 monostearate and PEG 1750 monostearate), and derivatives thereof. Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a polyoxyl castor oil, or derivative thereof. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate is a mixture of approximately 75% relatively hydrophobic of which a major portion is glycerol polyethylene glycol 12-oxystearate.

When preparing a formulation of the extracted prenylflavonoid such as xanthohumol and non-ionic surfactant, typically, these two ingredients can be present at a weight ratio is from about 1:5 to about 1:200, though ratios outside of this range can also be used. Thus, in the water-soluble concentrate or gel, these two ingredients may be the only two ingredients present, and they can be present within this ratio range. If admixed with a solid of liquid carrier and/or other excipients, this ratio can remain the same, such as when water is added to form a liquid spray, gel formulation, beverage, or a solid carrier is added to form a tablet or capsule or food supplement.

For the preparation of consumable formulations from the pharmaceutical formulations, pharmaceutically acceptable carriers can be either solid or liquid. Solid form preparations include powders, tablets, bilayer tablets or capsules, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the prenylflavonoid or xanthohumol mixed with a filler or binders such as lactose or starches, lubricants such as talc or magnesium stearate, and, optionally, stabilizers. In soft capsules, the prenylflavonoid or xanthohumol compounds may be dissolved or suspended in suitable liquids, such as fatty oils, surfactants, non-ionic surfactants, liquid paraffin, or liquid polyethylene glycol with or without stabilizers.

After addition of the carrier to the pharmaceutical formulation, the consumable formulation may take the form of a water-soluble formulation such as a spray, gel, mouthwash, mouth-rinse, or a beverage formulation, a food, formulation, a capsule formulation, a tablet formulation, and any combination thereof.

In some embodiments, the water-soluble formulation for administration can be a water solubilized formulation. A “water solubilized formulation,” as used herein, includes a prenylflavonoid such as xanthohumol, and a non-ionic surfactant, and water (e.g. a water containing liquid) but does not necessarily include organic solvents (e.g. ethanol). In some embodiments, the water solubilized formulation is a transparent water-soluble formulation.

In one aspect, the present disclosure provides a water-soluble formulation for administration that comprises or consists essentially of a prenylflavonoid, such as xanthohumol, and a non-ionic surfactant, and optionally water and/or excipients. In some embodiments, the water-soluble formulation does not include a vegetable oil suspension or visible macro-micelles (micelles visible to the naked eye) in water. In other embodiments, the water-soluble formulation does not include an alcohol (e.g. the compound is not first dissolved in alcohol and then added to water). In another aspect, a higher concentration of the extract is preferred due to a higher concentration of the active compound in the liquid formulation.

In some embodiments, the water-soluble formulation for administration includes the prenylflavonoid compound, or xanthohumol, and polyoxyl castor oil to form a transparent water-soluble formulation. In certain embodiments, light may be transmitted through the transparent water-soluble formulations without diffusion or scattering. Thus, in some embodiments, the transparent water-soluble formulations are not opaque, cloudy or milky-white. Transparent water-soluble formulations disclosed herein do not include milky-white emulsions or suspensions in vegetable oil such as coconut oil or other vegetable oils. Transparent water-soluble formulations are also typically not formed by first dissolving the compound in alcohol, and then mixed with water.

In some embodiments, a water-soluble formulation for administration can be in the form of a pharmaceutical composition or beverage. The pharmaceutical composition may include a prenylflavonoid such as xanthohumol, extracted from hops, and a non-ionic surfactant, and a pharmaceutically acceptable excipient, or water. After a pharmaceutical composition of the invention has been formulated in an acceptable carrier, it can be placed in an appropriate container and labeled for treatment of an indicated condition.

In such embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of xanthohumol is present in the water-soluble formulation. In other embodiments, 0.1 mg to 2 g, 0.5 mg to 1 g, 1 mg to 500 mg, 1 mg to 100 mg, 1 mg to 50 mg, 1 mg to 10 mg, or 1 mg to 5 mg of xanthohumol is present in the water-soluble formulation.

Any appropriate dosage form is useful for administration of the water-soluble formulation of the present invention, such as any oral or nasal dosage forms. Oral preparations include sprays, beverages, lozenges, tablets, pills, powder, dragees, capsules (e.g. soft-gel capsules), liquids, lozenges, gels, syrups, slurries, suspensions, bulb, inhaler, sprayer, nebulizer, or mask, suitable for making contact with the receptor in the oral or nasal cavity of the patient.

In dietary compositions for administration, especially in food, sprays, and beverages for humans, the prenylflavonoid or xanthohumol or any mixture of prenylflavonoids is suitably present in an amount in the range of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage. Beverages encompass non-alcoholic and alcoholic drinks as well as liquid preparations to be added to drinking water and liquid food. Non-alcoholic drinks are e.g. soft drinks, sport drinks, fruit juices, lemonades, near-water drinks (i.e. water-based drinks with low calorie content), teas and milk-based drinks. Liquid foods are e.g. soups and dairy products.

Xanthohumol may also be present, for example, in a fast dissolving tablet formulation or fast dissolving strip delivery system, at a concentration from 0.5 to 50 mg per tablet combined with a suitable disintegrating agent. In other embodiments, the xanthohumol is present at a concentration from 0.01 mg/ml to 25 mg/serving in a tablet, capsule, spray, mouth rinse, strip, or beverage. The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The quantity of active component in a unit dose preparation may be varied or adjusted according to the particular application and the potency of the active component. The composition can, if desired, also contain other compatible therapeutic agents.

As mentioned, when preparing a suitable formulation, any formulation that effectively delivers the prenylflavonoid such as xanthohumol to the TAS2R38 receptor can be prepared. In one specific example, the xanthohumol can be prepared in a formulation by heating a water-soluble non-ionic surfactant in a container to a temperature of about 90° F. to about 200° F. while mixing the non-ionic surfactant until a clear non-ionic surfactant is formed; and adding the xanthohumol to the clear non-ionic surfactant and mixing until a clear non-ionic surfactant-xanthohumol combination is formed so as to constitute from about 20 wt % to 99.9 wt % surfactant and from 0.01 wt % to 10 wt % xanthohumol. The xanthohumol is thus sufficiently dispersed or dissolved in the surfactant so that a gel composition or emulsion is formed containing no visible micelles or particles of xanthohumol. The solution may be heated to increase solubility. The heating temperature is typically selected to avoid chemical breakdown of the non-ionic surfactant. Water can be used to solvate the gel or concentrate of the emulsion to make a beverage in one example, or can be fortified directly (with or without added water) into gels, lozenges, capsules, creams, ointments, foods, etc.

In one specific embodiment, the temperature of both can be maintained at from 90 and 150° F. In some embodiments, the resulting solution is a water-soluble formulation or transparent water-soluble formulation as described above. For example, the resulting solution may be a water-soluble formulation that is a crystal-clear solution, with no particles visible to the naked eye. Alternatively, the gel composition (prior to addition with water) will be combinable with warm water, as described above, to form a water-soluble formulation.

In another aspect, the disclosure relates to the use of an effective amount of a prenylflavonoid, e.g., xanthohumol, or any mixture thereof, for the manufacture of a composition for the treatment of upper respiratory infections, or prevention of infection by various pathogens. In another aspect, the disclosure relates to the use of an effective amount of a prenylflavonoid, e.g., xanthohumol, or any mixture thereof for increasing lifespan or longevity. The invention relates to the use of an effective amount of a prenylflavonoid, such as xanthohumol, or any mixture thereof, to treat or prevent upper respiratory infections from viruses, bacteria, fungi or other invading microorganisms or pathogens. The disclosure also relates to the use of an effective amount of hops xanthohumol, or any mixture thereof, formulated with a non-ionic surfactant, or other solubilizing agent, for the manufacture of a water-soluble, composition for the treatment or prevention of invading pathogens such as bacteria, viruses, or fungi. In addition, the present invention is also directed to a method for lengthening the lifespan of a subject, said method comprising administering an effective dose of a prenylflavonoid, such as xanthohumol or any mixture thereof to animals or humans which are in need thereof. In this regard an effective dose of xanthohumol, may especially be used for stimulating, activating, or up regulating the specific bitter taste receptor TAS2R38, by acting as a specific agonist to that receptor. Other therapeutic benefits may be discovered in the future related to stimulation of the TAS2R38 bitter taste receptor, including the presence of the receptor in other parts of the body (non-oral or nasal).

The amount of xanthohumol sufficient to have a therapeutic effect on a subject with an affective mood disorder condition may be from about 0.5 mg to about 1000 mg, from about 1 mg to about 50 mg, from about 1 mg to about 20 mg, or about 3 mg to about 10 mg. In some embodiments, the dose of xanthohumol is 1 mg, 3 mg, 5 mg, 10 mg, or 20 mg. or 50 mg. In still other embodiments, the dose of xanthohumol is about 5 mg. The xanthohumol is typically administered as a twice per day formulation or as a once per day formulation.

In solid dosage unit preparations for humans, xanthohumol is suitably present in an amount in the range of from about 0.1 mg to about 1000 mg, preferably in the range of from about 1 mg to about 500 mg per dosage unit. More preferably, in a range of about 1 mg to about 100 mg. Dosages within these ranges can be relevant to both consumable compositions and mouth-sprays, gels, or rinses, and can be modified within appropriate ranges as would be appreciated by one skilled in the art after considering the present disclosure.

In dietary compositions, especially in food and beverages for humans, the hops xanthohumol is suitably present in an amount in the range of from about 0.0001 (1 mg/kg) to about 5 weight-% (50 g/kg), preferably from about 0.001% (10 mg/kg) to about 1 weight %, (10 g/kg) more preferably from about 0.01 (100 mg/kg) to about 0.5 weight-% (5 g/kg), based upon the total weight of the food or beverage. In food and drinks, the range is from 5 to 50 mg per serving, i.e. about 120 mg per kg food or drink. The amount of turmeric extract or curcumin in an amount in the range of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage. In food and drinks, the range is from 5 to 500 mg per serving.

For animals, excluding humans a suitable daily dosage of xanthohumol, may be within the range of from 0.001 mg per kg body weight to about 1000 mg per kg body weight per day. More preferred is a daily dosage in the range of from about 0.1 mg to about 500 mg per kg body weight, and especially preferred is a daily dosage in the range of from about 1 mg to 50 mg per kg body weight. be within the range of from 0.001 mg per kg body weight to about 100 mg per kg body weight. A preferred serving size of a suitable mouth rinse, spray, lozenge, rinse, beverage, or instantly dissolvable tablet would contain a minimum of about 1 mg. xanthohumol or prenylflavonoid per serving or application.

A prenylflavonoid formulation such as xanthohumol may be incorporated into various types of dosage forms for convenient consumption by animals or humans. Examples of fortified foods are cereal bars and bakery items such as cakes and cookies. Additionally, the combination can be administered in the form of a beverage, food, feed, dairy product, yoghurt, fortified food, enhanced water, cereal bars, bakery item, cake, cookies, dietary supplement, tablet, pill, granules, dragees, capsules, effervescent formulations, non-alcoholic drinks, sprays, soft drinks, sport drinks, fruit juices, teas, milk-based drinks, liquid foods, soups, liquid dairy products, or any combination thereof.

The foregoing detailed description describes the disclosure with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

### EXAMPLES

The xanthohumol used in the following Examples was extracted from hops flowers and was analyzed by HPLC.

### Formulations

Water soluble compositions of xanthohumol were formulated containing polyoxyl castor oil. The polyoxyl castor oil (non-ionic surfactant) was heated and stirred to a temperature of about 150° F. The powdered xanthohumol (a hops extract containing 42% xanthohumol) was added slowly and mixed until a clear viscous solution was formed containing dissolved xanthohumol (hereinafter referred to as “the emulsion phase,” “gel,” or “water-soluble concentrate”). The xanthohumol/surfactant mixture was then slowly added to warm water (120° F.) until a crystal-clear solution was formed. The resulting concentration of xanthohumol in the clear water-soluble liquid solution was 7 mg/ml. (Table 1).

TABLE 1

| Ingredient | V % |
|---|---|
| Xanthohumol Extract (42%) | 1.3 |
| Water | 73.20% |
| Polyoxyl Castor Oil | 25% |
| Sodium Benzoate | 0.06% |
| Potassium Sorbate | 0.04% |
| Citric Acid | 0.4% |
| Total | 100% |

A stock solution containing 1 μM xanthohumol was prepared for gene expression research.

### Example 1

In an effort to determine the activity of xanthohumol on gene expression, the following study was conducted.

### Objective

The objective of this study was to determine the TAS2R38 gene activity of xanthohumol at a concentration of 1 μM.

### Experimental Procedure

If the sinonasal immune defense is impaired, diseases such as chronic rhinosinusitis or CRS may occur. CRS (chronic rhinosinusitis) is a fairly common disease, affecting more than 16 million Americans annually, and results in about 25% of all adult antibiotic prescriptions. Inhaled pathogens, such as bacteria and viruses, toxins, and particulates continuously challenge the respiratory system. The principal physical defense against these inhaled insults is mucociliary clearance (MCC), which has 2 components, mucus production and mucus transport. Coordinated ciliary beating transports debris-laden mucus from both the upper and lower respiratory passages toward the oropharynx, from which it is cleared by expectoration or swallowing. Ciliary beating accelerates in response to multiple host and environmental stimuli through several second messenger pathways including intracellular Ca2+ and NO production. In addition to its role as a second messenger, NO diffuses into the airways where it has antimicrobial properties and is therefore central to host defense against respiratory infections.
The pathogen-derived triggers of respiratory host defenses are likely to include those that humans report to taste bitter, based on recent observations that bitter taste receptors (T2Rs) are expressed in both upper and lower human respiratory epithelium.
Additionally, in vitro stimulation of human lower airway cultures with various bitter-tasting compounds increased intracellular Ca2+ and ciliary beat frequency (CBF).
Preliminary screening studies identified prominent expression of T2R38, the receptor for the compound phenylthiocarbamide (PTC), in upper airway epithelium. Bitter receptors, and particularly T2R38, are unique among G protein-coupled receptors in the density of their naturally occurring genetic variants. Many previous studies have demonstrated that people with the taster form of this receptor report that concentrations of some bitter ligands like PTC are intensely bitter at concentrations that are imperceptible to those with the non-taster form. Functional differences in taste perception predict functional responses in the airway to pathogen-derived quorum-sensing molecules. If true, genetically determined individual taste perception could identify individuals susceptible to bacterial respiratory infections and may be used to guide alternative therapeutic interventions.
The activity and functionality of taste receptor modulation can be measured in primary human sinonasal cells genotyped for TAS2R38 (encoding T2R38) and cultured at an air-liquid interface (ALI), duplicating the polarized respiratory epithelium. ALI cultures are a state-of-the-art epithelial model that has been used for extensive studies of the upper and lower airways. Three indexes of respiratory innate defense can be measured, NO production, mucociliary clearance, and bactericidal activity. It has been found that T2R38 plays a critical role in the detection and response to quorum-sensing molecules produced by gram-negative bacteria, including the important respiratory pathogens such as Pseudomonas aeruginosa. Differences in T2R38 functionality, as assessed by TAS2R38 genotype, significantly correlated with differential susceptibilities of patients to gram-negative bacterial sinonasal infection.
It is clear that human taste receptor T2R38 is expressed and functional in human upper airway cells, and that taste receptors are intimately involved in upper airway innate immunity and immune detection of infection.
In an effort to understand the activity of xanthohumol on taste receptors, a Gene Expression and DNA Microarray Study was conducted to determine which genes might be up-regulated or down-regulated by the compound, and if any of these genes are associated with taste receptors. Human keratinocytes were used, as the TAS2R38 gene is expressed and functional in these cells.

### Experiment 1; DNA Microarray Study

### Summary of Test Methods

The testing model used in this study is the MatTek full thickness tissue (EFT-200). This skin model consists of normal human-derived epidermal keratinocytes that have been cultured to form a multilayered, highly differentiated model of the human epidermis and human fibroblasts that have been seeded into a collagen matrix to form the dermis. This model also introduces a well-defined basement membrane between the epidermal and dermal layers, which is essential to enable in vivo like interactions between the keratinocytes and the fibroblasts.

### Tissue Preparation

Upon arrival, the MatTek ET-200 tissue was stored at 4° C. until used (they can be stored for up to 72 hours however tissue viability may decrease with prolonged storage). Prior to use, the tissues to be used were removed from the agarose-shipping tray and placed into a 6-well plate containing 2.0 ml of assay medium (37±2° C.). All of the agarose must be removed from the outside of the tissue culture insert since any residual agarose may prevent the assay medium from reaching the tissue. The tissues were allowed to incubate for at least 24 hours at 37±2° C. and 5±1% CO₂. After this initial incubation, the assay medium was replaced with 5 ml of fresh medium (37±2° C.).

### Preparation of Test Materials

Test materials were prepared at the required dilutions using phosphate buffered saline (PBS) as the diluent.

### Application of Test Material

Fifty (50) μl or mg of test material was applied directly onto the surface of the tissue. The 6-well plates will then be incubated at 37±2° C. and 5±1% CO₂ for 48 hours (the positive control was supplemented with fresh sodium ascorbate at 24 hours of incubation due to the short half-life of sodium ascorbate).

### Tissue/Culture Media Collection

At the end of the incubation any residual test material was removed from the tissues by rinsing them twice with at least 100 μl of PBS. The tissue culture medium was collected and stored at −20° C. until assayed.
Since human keratinocytes express functional bitter taste receptor TAS2R38, this is a good tissue to look for activity.
DNA microarrays were used to screen xanthohumol, a representative prenylflavonoid derived from hops, for changes in the expression of thousands of different genes. DNA microarrays are extremely powerful tools that allow users to analyze changes in gene expression by monitoring changes in the messenger ribonucleic acid (“mRNA”) of hundreds to thousands of genes in a single experiment. All cells function by using their genes to make protein products. This process starts by making an mRNA copy of the gene through a process called transcription. The mRNA copy is then translated into a protein that plays a functional role within the cell or the cell's environment. Since the process of gene expression is highly regulated, the amount of mRNA can be a good indicator of the level of activity for a specific gene. With the introduction of DNA microarrays, researchers can now rapidly obtain a much more global view of what is happening inside of a cell since the results of one or two array experiments can potentially generate data on changes in gene expression across the entire known human genome.
If a treatment does not exert an effect on a gene, then the median fluorescent intensity of the feature corresponding to that specific gene will be the same for both the scan from a red laser and the scan from a green laser. This would result in a ratio of 1. If a treatment increases the expression of a gene, then the median intensity of the feature would be greater in the scan from the red laser than the green, resulting in a ration that is greater than 1. A ratio that is less than 1 would, therefore, indicate that the treatment reduced the expression of a gene. This is called the “ratio of means.” In the present data, a ratio of greater than or equal to 1.3 as the cut-off for up-regulated genes and a ratio of less than or equal to 0.7 as the cut-off for down-regulated genes was used. These broader ranges are a more conservative judging criterion and account for possible sources of variation in fluorescence intensity that are not associated with the treatment. By providing information on how active ingredients affect systems of immediate interest, the data from microarray experiments can provide a valuable library that catalogs in detail the effects of a specific material which can be reviewed at a future time. The array will also have data on enzymes involved in lipid metabolism, DNA repair enzymes which can be a factor in ageing, and antioxidant enzymes.
In addition to the information on active ingredients and raw materials that the DNA microarray can provide, the arrays can be used to better characterize the mechanisms of action related to certain therapeutic categories. The array data also allows the verification that a gene of interest is expressed in a certain model before running an experiment. The following tables provide the data based on the ratio of means after 504, (0.05 ml) of a solubilized xanthohumol liquid formulation with a concentration of 5 mg/ml xanthohumol was used in the following experiment. RNA was isolated from the tissues for subsequent analysis via DNA microarrays.

### Results of Full Thickness Tissue DNA Microarray Screening

Up-regulation of TAS2R38 and TAS2R16 Genes, Ratio of Means

| | Ratio of Medians | Gene Name | Description |
|---|---|---|---|
| NM_176817 | 21 | TAS2R38 | Homo sapiens taste |
| | | | receptor, type 2, |
| | | | member 38 MRNA |
| NM_016945 | 4.5 | TAS2R16 | Homo sapiens taste |
| | | | receptor, type 2, |
| | | | member 16, MRNA |

As described above, a gene is up-regulated if the ratio of means is above 1.3, and down-regulated if the ratio of means is below 0.7. As can be seen from the data, both the TAS2R38 and the TAS2R16 genes were significantly up-regulated, with TAS2R38 up-regulated with a ratio of medians of 21. The magnitude of this up-regulation is very unusual and makes it stand out among all the taste receptor genes. The TAS2R38 gene is the Homo sapiens taste receptor, type 2, member 38 mRNA (NM_176817), and the TAS2R16 gene is the Homo sapiens taste receptor mRNA (NM_016945).

Given the newly discovered and previously unknown ability of the prenylflavonoid xanthohumol to activate the TAS2R38 and TAS2R16 taste receptors in functional human keratinocytes, this molecule could be used to formulate various forms of oral products designed to stimulate the innate immune system. In particular, oral products such as oral sprays, inhalers, nebulizers, masks, lozenges, drinks, gums, gels, or other consumables that provide a vehicle to make contact with the oral mucosa or nasal passages have been formulated. All of these treatment forms or products require solubilized forms of the molecule, or forms that will become soluble in saliva. A “non-ionic surfactant,” as used herein, is a surface-active agent that tends to be non-ionized (i.e. uncharged) in neutral solutions (e.g. neutral aqueous solutions). Useful non-ionic surfactants include, for example, non-ionic water-soluble mono-, di-, and tri-glycerides; non-ionic water soluble mono- and di-fatty acid esters of polyethyelene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as poloxamer 406 (PLURONIC F-127), and derivatives thereof. Examples of non-ionic water soluble mono-, di-, and tri-glycerides include propylene glycol dicarpylate/dicaprate (e.g. Miglyol 840), medium chain mono- and diglycerides (e.g. Capmul and ImwitoR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), and derivatives thereof.
Non-ionic water-soluble mono- and di-fatty acid esters of polyethyelene glycol include d-alpha-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof. Natural non-ionic surfactants such as saponins derived from Quillaja saponaria tree bark may be used.
Polyglycolyzed glycerides include polyoxyethylated oleic glycerides, polyoxyethylated linoleic glycerides, polyoxyethylated caprylic/capric glycerides, and derivatives thereof. Specific examples include Labrafil M-1944CS, Labrafil M-2125CS, Labrasol, SOFTIGEN, and GELUCIRE.
In some embodiments, the non-ionic surfactant is a polyoxyl castor oil, or derivative thereof may be used to solubilize the prenylflavonoid. In some embodiments, a natural non-ionic surfactant such as saponins derived from Quillaja saponaria tree bark may be used. Naturally derived surfactants such as saponins from Quillaja saponaria tree Bark, soapwort (Gypsophila Sp.) extracts, and chestnut saponin extracts may be used as solubilizing agents.
Any solubilizing system that is capable of increasing the solubility of a lipophilic compound may be employed, such as saponins, cyclodextrins, phospholipids, hydrocolloids, proteins, polysaccharides, rhamnolipids, sophorolipids, or even alcoholic formulations. High pressure (microfluidizer) or ultrasonic homogenization may be used. Stable nanoemulsions may be employed to make gels, some containing water, others with no or very little water.
Activation of specific taste receptors such as TAS2R38 represents a very different means of stimulating the innate immune system than suppression of the proliferation of T cells, generation of cytotoxic effector cells, or production of cytokines, all mediated by inhibition of NF-kappa B responsive genes. This new immunomodulatory activity is independent of inhibition of T cell proliferation, cell mediated cytotoxicity and Th1 cytokine production through suppression of NF-kappa-B. The discovery of this novel mechanism for the action of xanthohumol on the innate immune system via taste receptors is independent of suppression of NF-kappa-B and represents a more rapid and sentry-based mode of action, a more direct means of inactivating pathogens prior to systemic downstream modulation the immune system. This upfront sentry system for the prevention of the entry of pathogens, or the rapid inactivation of invading organisms represents an entirely new method for protection of the respiratory system at the entry point, the mouth and nasal passages. It represents a pre-systemic treatment modality and does not require entry of the molecule into the blood stream, via presentation to the liver (no first pass metabolism). Therefore, it is also not subject to systemic metabolism, but functions through direct contact of the agonist molecule with the taste receptor prior to ingestion.

### Example 2

### qPCR (Polymer Chain Reaction) Study

Currently, very little is known about compounds that directly agonize and regulate TAS2R38 expression. Because of this, research related to the prenylflavonoid xanthohumol's effects on gene expression was compared with other TAS2R38 targeting compounds, namely sinigrin, a glucosinolate connected to the family of glucosides found in the Brassicaceae vegetable family and found in foods such as brussels sprouts and broccoli. Sinigrin selectively triggers the taster variant of the TAS2R38 receptor (Meyerhof, W. et al. The Molecular Receptive Ranges of Human TAS2R Bitter Taste Receptors. Chem Sense (2010) 35: 157-70.) Sinigrin was used as a comparator at a concentration of 1 the same concentration as xanthohumol (1 μM).

Two hTBEC cultures were selected for this initial study. hTBEC 1 is derived from one of the most bitter-responsive cultures, which has a moderate/moderately low basal gene expression level of TAS2R38. hTBEC 2 is derived from a culture that responds to bitter stimuli, but has a high response in particular to TAS2R38 expression compared with other hTBEC repositories. RNA was harvested from cultures treated cultures and control cultures at 1 μM lead compound at 24 and 48 hours post treatment. Following is a table that explains the experimental paradigm in full:

| | 24 Hour | | | 48 Hour | | |
|---|---|---|---|---|---|---|
| hTBEC 1 | CTR-No | CTR-No | CTR-No | CTR-No | CTR-No | CTR-No |
| | Treatment | Treatment | Treatment | Treatment | Treatment | Treatment |
| hTBEC 2 | CTR-No | CTR-No | CTR-No | CTR-No | CTR-No | CTR-No |
| | Treatment | Treatment | Treatment | Treatment | Treatment | Treatment |
| hTBEC 1 | xanthohumol | xanthohumol | xanthohumol | xanthohumol | xanthohumol | xanthohumol |
| | 1 μM | 1 μM | 1 μM | 1 μM | 1 μM | 1 μM |
| hTBEC 2 | xanthohumol | xanthohumol | xanthohumol | xanthohumol | xanthohumol | xanthohumol |
| | 1 μM | 1 μM | 1 μM | 1 μM | 1 μM | 1 μM |
| hTBEC 1 | Sinigrin | Sinigrin | Sinigrin | Sinigrin | Sinigrin | Sinigrin |
| | 1 μM | 1 μM | 1 μM | 1 μM | 1 μM | 1 μM |
| hTBEC 2 | Sinigrin | Sinigrin | Sinigrin | Sinigrin | Sinigrin | Sinigrin |
| | 1 μM | 1 μM | 1 μM | 1 μM | 1 μM | 1 μM |

Note:
N = 3 biological replicates, N = 2 experimental replicates for each qPCR reaction (i.e. 6 ddCT values will be obtained per each treatment condition). The housekeeping gene β-actin was used.

In an effort to establishing a comprehensive understanding of xanthohumol's effects on gene expression, 3 unique primer pairs to accurately capture the TAS2R38 gene were developed as cultures. As demonstrated in many population genetics manuscripts, there are multiple sequence variants that give rise to differential responses to bitter stimuli in vivo. The published NCBI genetic sequence for the region of chromosome 7q that encodes TAS2R38 and overlay map the SNP variant markers to ensure the primers cover strategic regions of the TAS2R38 gene. For probing TAS2R38, 3 individual primer pairs were used for a comprehensive view of how the compound may be “hitting” the gene.

hTBEC 1 (most bitter responsive culture in functional assays). XN = xanthohumol

| | Tas2R38-5 | SD | Bactin | SD | Tas2R38C | SD | Bactin | SD | Tas2R38tb | SD | Bactin | SD |
|---|---|---|---|---|---|---|---|---|---|---|---|---|
| control 24 | 32.122 | 0.272 | 17.690 | 0.246 | 28.958 | 0.210 | 17.832 | 0.227 | 32.123 | 0.326 | 18.391 | 0.476 |
| XN 24 | 30.770 | 0.127 | 18.332 | 0.671 | 27.314 | 0.228 | 17.666 | 0.201 | 30.671 | 0.289 | 17.956 | 0.289 |
| sinigrin 24 hr | 32.238 | 0.272 | 17.998 | 0.427 | 29.116 | 0.150 | 18.024 | 0.155 | 32.123 | 0.326 | 18.374 | 0.524 |
| control 48 hour | 30.795 | 0.139 | 18.327 | 0.667 | 30.805 | 0.169 | 17.674 | 0.215 | 30.571 | 0.275 | 17.996 | 0.275 |
| XN 48 hour | 29.788 | 0.241 | 17.773 | 0.427 | 28.949 | 0.209 | 17.857 | 0.215 | 31.539 | 0.199 | 18.374 | 0.524 |
| sinigrin 48 hour | 30.270 | 0.127 | 17.841 | 0.368 | 30.027 | 0.204 | 17.674 | 0.215 | 30.596 | 0.311 | 17.996 | 0.311 |
| | Delta | Tas2R38 sdofdiff | 5prime ddCT | 2\hat{ }-ddct | Delta | Tas2R38C sdofdiff | DS ddCT | 2\hat{ }-ddct | Delta | Tas2R38 sdofdiff | 3prime ddCT | 2\hat{ }-ddct |
| control 24 | 14.432 | 0.366 | 0.000 | 1.000 | 11.126 | 0.309 | 0.000 | 1.000 | 13.732 | 0.577 | 0.000 | 1.000 |
| XN 24 | 12.438 | 0.683 | −1.994 | 3.983 | 9.648 | 0.303 | −1.478 | 2.785 | 12.714 | 0.409 | −1.017 | 2.024 |
| sinigrin 24 hr | 14.240 | 0.506 | −0.192 | 1.142 | 11.093 | 0.216 | −0.033 | 1.023 | 13.748 | 0.617 | 0.017 | 0.988 |
| control 48 hour | 12.468 | 0.681 | 0.000 | 1.000 | 13.131 | 0.273 | 0.000 | 1.000 | 12.574 | 0.389 | 0.000 | 1.000 |
| XN 48 hour | 12.016 | 0.490 | −0.452 | 1.368 | 11.093 | 0.300 | −2.039 | 4.109 | 13.165 | 0.561 | 0.591 | 0.664 |
| sinigrin 48 hour | 12.430 | 0.389 | −0.038 | 1.027 | 12.353 | 0.296 | −0.779 | 1.716 | 12.599 | 0.441 | 0.025 | 0.983 |

hTBEC 2 (highest basal tas2r38 expression among hTBEC cultures)

| | Tas2R38-5 | SD | Bactin | SD | Tas2R38C | SD | Bactin | SD | Tas2R38tb | SD | Bactin | SD |
|---|---|---|---|---|---|---|---|---|---|---|---|---|
| control 24 | 28.012 | 0.282 | 19.273 | 0.463 | 26.525 | 0.195 | 19.365 | 0.158 | 28.427 | 0.295 | 19.957 | 0.548 |
| XN 24 | 27.214 | 0.113 | 19.859 | 0.266 | 25.495 | 0.295 | 19.166 | 0.233 | 26.977 | 0.311 | 19.516 | 0.311 |
| sinigrin 24 hr | 27.234 | 0.221 | 17.879 | 0.429 | 25.730 | 0.204 | 17.964 | 0.216 | 27.722 | 0.218 | 18.515 | 0.510 |
| control 48 hour | 27.021 | 0.117 | 18.451 | 0.683 | 27.052 | 0.182 | 17.780 | 0.216 | 26.705 | 0.484 | 18.104 | 0.484 |
| XN 48 hour | 27.026 | 0.229 | 17.776 | 0.427 | 25.038 | 0.193 | 17.827 | 0.170 | 26.985 | 0.236 | 18.378 | 0.491 |
| sinigrin 48 hour | 26.389 | 0.337 | 18.215 | 0.556 | 27.460 | 0.190 | 17.511 | 0.241 | 26.075 | 0.341 | 17.999 | 0.341 |
| | Delta | Tas2R38 sdofdiff | 5prime ddCT | 2\hat{ }-ddct | Delta | Tas2R38C sdofdiff | DS ddCT | 2\hat{ }-ddct | Delta | Tas2R38 sdofdiff | 3prime ddCT | 2\hat{ }-ddct |
| control 24 | 8.739 | 0.542 | 0.000 | 1.000 | 7.161 | 0.251 | 0.000 | 1.000 | 8.470 | 0.622 | 0.000 | 1.000 |
| XN 24 | 7.356 | 0.288 | −1.383 | 2.609 | 6.329 | 0.376 | −0.831 | 1.779 | 7.461 | 0.439 | −1.009 | 2.013 |
| sinigrin 24 hr | 9.355 | 0.483 | 0.616 | 0.553 | 7.766 | 0.297 | 0.605 | 0.657 | 9.207 | 0.555 | 0.736 | 0.600 |
| control 48 hour | 8.571 | 0.693 | 0.000 | 1.000 | 9.272 | 0.283 | 0.000 | 1.000 | 8.601 | 0.685 | 0.000 | 1.000 |
| XN 48 hour | 9.250 | 0.484 | 0.679 | 0.624 | 7.210 | 0.257 | −2.062 | 4.175 | 8.607 | 0.545 | 0.007 | 0.995 |
| sinigrin 48 hour | 8.173 | 0.650 | −0.397 | 1.317 | 9.949 | 0.307 | 0.677 | 0.625 | 8.077 | 0.482 | −0.524 | 1.436 |

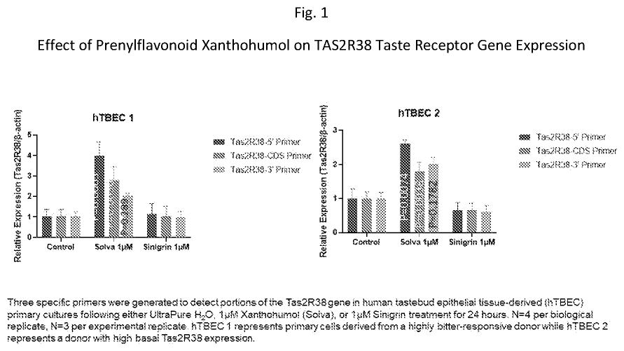

As can be seen from the results of the study graph in FIG. 1, the water-soluble xanthohumol formulation at a concentration of 1 μM resulted in significant up regulation of the TAS2R38 gene (approximately 4× higher than baseline or the Sinigrin control at 1 μM) within 24 hours.

While the above examples are illustrative of the principles and concepts discussed herein, it will be apparent to those of ordinary skill in the art that numerous modifications in form, usage and details of implementation can be made without the exercise of inventive faculty, and without departing from those principles and concepts. Accordingly, it is not intended that the principles and concepts be limited, except as by the claims set forth below.

What is claimed is:

1. A method of increasing the activity of the TAS2R38 bitter taste receptor gene comprising a prenylflavonoid.

2. The method of claim 1, wherein the prenylflavonoid is xanthohumol.

3. The method of claim 1, wherein the xanthohumol is present at a concentration from about 0.01% to 50% by weight.

4. The method of claim 1, wherein the formulation further comprises an emulsifier such as a non-ionic surfactant, ethanol, d-alpha tocopheryl polyethylene glycol succinate, glycerin, cycoldextrin, phosphatidylcholine, medium chain triglycerides, hydrocolloids, naturally derived surfactants such as saponins from Quillaja Bark, soapwort (Gypsophila Sp.) extracts, and chestnut saponin extract, PEGylated nanospheres, PLGA nanospheres, or nanoparticles.

5. The method of claim 4, wherein the non-ionic surfactant is selected from the group comprising non-ionic water-soluble mono-, di-, or tri-glycerides; non-ionic water soluble mono- or di-fatty acid esters of polyethylene glycol; non-ionic water soluble sorbitan fatty acid esters; polyglycolyzed glycerides; non-ionic water soluble triblock copolymers; their derivatives; and combinations thereof.

6. The method of claim 5, wherein the non-ionic surfactant is a non-ionic water-soluble mono-, di-, or tri-glyceride.

7. The method of claim 6, wherein the non-ionic surfactant is polyoxyl castor oil.

9. The method of claim 5, wherein the xanthohumol to non-ionic surfactant weight ratio is from about 1:5 to about 1:200.

10. A method of stimulating upper airway immunity and increasing lifespan in a human or animal comprising administering a prenylflavonoid that is an agonist of the TAS2R38 receptor.

11. The method of claim 10, wherein the prenylflavonoid is xanthohumol.

12. The method of claim 10, further comprising a solubility enhancing agent, wherein the solubility enhancing agent is an emulsifier such as, ethanol, non-ionic surfactants, d-alpha tocopheryl polyethylene glycol succinate, naturally derived surfactants such as saponins from Quillaja Bark, soapwort (Gypsophila Sp.) extracts, and chestnut saponin extracts glycerin, cyclodextrin, phosphatidylcholine, medium chain triglycerides, hydrocolloids, PEGylated nanospheres, PLGA nanospheres, or nanoparticles.

13. The method of claim 12, wherein the formulation is in the form of a water-soluble gel or liquid concentrate.

14. The method of claim 10, wherein the formulation is applied to the mouth or nasal passages as a spray, mouth-rinse, tincture, or beverage formulation.

15. The method of claim 1, wherein the prenylflavonoid is administered as a consumable formulation selected from a group comprising sprays, mouth rinses, lozenges, rapidly dissolving strips, fast dissolving tablets, bulb, inhaler, sprayer, nebulizer, mask, beverages, food, feed, dairy products, yoghurts, fortified food, enhanced waters, cereal bars, bakery items, cakes, cookies, dietary supplements, tablets, pills, granules, dragees, capsules, effervescent formulations, non-alcoholic or alcoholic drinks, soft drinks, sport drinks, fruit juices, teas, milk-based drinks, liquid foods, soups, liquid dairy products, and any combination thereof.

16. A method of treating respiratory infection, chronic rhinosinusitis or rhinovirus, or severe acute respiratory syndrome coronavirus or SARS-CoV-2 virus, or variants of the virus, in subjects expressing the homozygous AVI/AVI polymorphism or heterozygous PAV/AVI alleles of the TAS2R38 taste receptor, by contacting the oral cavity or the sinonasal area with a soluble formulation of a prenylaflavonoid.

17. The method of claim 16, wherein the prenylflavonoid comprises xanthohumol.

18. The method of treating or preventing the SARS-CoV2 virus or variants of the virus from infecting the upper and lower human airway epithelium, by boosting the host defense against respiratory infections using the prenylflavonoid xanthohumol in an oral or nasal formulation comprising a solubilized gel emulsion, oral rinse, liquid solution, bulb, inhaler, sprayer, nebulizer, or mask.
